(19) United States
(12) Patent Application Publication
(10) Pub. No.: US 2023/0270590 A1
(43) Pub. Date: Aug. 31, 2023

(54) GEL CAP

(71) Applicant: Leticia V. Mussi Ross, San Dieg, CA (US)

(72) Inventor: Leticia V. Mussi Ross, San Dieg, CA (US)

(21) Appl. No.: 18/144,359

(22) Filed: May 8, 2023

(51) Int. Cl. A61F 7/02; A61F 7/00

(52) U.S. Cl. CPC A61F 7/02; A61F 2007/0007; A61F 2007/0004; A61F 2007/0219; A61F 2007/0008; A61F 2007/0015; A61F 2007/0268

(57) ABSTRACT

Presented is a gel cap adapted to relieve headaches, migraine, headache, fever, stress, or similar medical conditions in users. The gel cap includes one or more chambers defined by one or more stretchable and flexible pieces of fabric, and filled with a temperature-variable substance. In use, one of the chambers substantially covers a frontal region of a user's head and a front face extending until the cheekbones of the user, and another chamber substantially covers a rear region of the user's head extending from parietal to occipital region. The chambers are isolated from each other by a region extending from mesh covers that attaches one chamber to the other chamber and substantially cover ears of the user. The chamber covering frontal region of the user's head and the front face includes a V shaped cut-out section conforming to the nose of the user.

### TECHNICAL FIELD

The present invention relates to headwear, more particularly the present invention relates to a gel cap that can be worn over the forehead or pulled down to cover the entire face and head of a user leaving no or minimal area untreated during cold or hot therapy for providing relief from headaches, migraine, headache, fever, stress or similar medical conditions. The gel cap further embodies mesh wall coverings on either side to cover the ears of the user.

### BACKGROUND

The causes of headaches, fever, migraine, stress, or other similar medical conditions are not clearly known. Headaches or migraine are believed to be caused by surrounding tissues, brain chemicals, blood vessels, and nerves that produce pain signals. One very well-known treatment for migraine or headaches is applying hot or cold compresses to the head or neck areas of the user. Widely used ice packs usually dull the sensation of pain and heat packs or heating pads usually relax tense muscles.

Icepacks having flexible bags have traditionally been used to hold ice and/or frozen water. These bags are generally used for helping with headaches and other medicinal purposes. However, as a headache or migraine remedy, these traditional icepack bags are difficult to position and often do not stay in place on one's head. These ice packs in the form of bags are inherently not designed to fit the head of the user. As a headache/migraine remedy, the bag must be continuously balanced on the patient's head not allowing the patient to move or lie down. This constant balancing further inhibits the user from applying the bag and ice contents to selected and needed locations on one's head.

Thus, there is a continuing need for new devices and methods for treating or alleviating the symptoms associated with headaches, migraine, headaches, fever, and stress. The inventor herein proposes a novel gel cap that can be worn over a forehead or pulled down to cover the entire face of the user to selectively apply hot and cold presses at different points around the head to provide relief from headaches, migraine, headache, fever, stress or similar medical conditions.

### SUMMARY

Accordingly, there is provided embodiments related to a gel cap wearable by a user. The gel cap for the user's head allows applications (of hot or cold therapy) and coverage of all of the head regions and allows the user or patient to sit in any position, lie in any position or wear it during movement. Once worn, the gel cap is comfortably held in place allowing complete or localized cold or hot applications in a precise and steady manner.

The gel cap of the present invention can stretch to fit over different size heads of patients/users and can be used on the forehead or pulled down to cover the entire face of the user to effectively apply cold or hot therapy over various head points to relieve the user from headache or migraine or other similar medical conditions.

The gel cap of the present invention comprises one or more chambers filled with a temperature-variable substance (in gel form) that substantially surrounds the complete head portion of the user.

The gel cap of the present invention is effective in providing localized applications of cold or hot compresses on one's head.

According to an embodiment of the present invention, a gel cap includes a first chamber defined by a first stretchable piece of fabric, the first chamber is filled with a temperature variable substance, wherein, when in use, the first chamber substantially covers a frontal region of a user's head and a front face of a user extending until cheekbones of the user; and a second chamber formed by a second stretchable piece of fabric filled with the temperature variable substance, wherein, when in use, the second chamber substantially covers a rear region of the user's head extending from parietal to occipital region of the user's head. The first chamber further comprises a cut-out section conforming to the nose of the user so as not to obstruct the breathing of the user. The first chamber and the second chamber along with the temperature variable substance are curved and streamlined so that the gel cap fits better around the front face of the user extending until the cheekbones and around the rear region of the head.

According to the same embodiment, the first chamber is isolated from the second chamber by a region having a predefined width (W) extending from a first mesh cover to a second mesh cover, wherein each of the first and second mesh covers attaches the first chamber to the second chamber and substantially cover ears of the user on either side.

In an embodiment, the temperature variable substance is a malleable soft-solid gel.

In an embodiment, the first stretchable piece of fabric and the second stretchable piece of fabric are identical or unidentical material.

In an embodiment, the first stretchable piece of fabric (101a) and the second stretchable piece of fabric is a single one-piece fabric.

In an embodiment, the first stretchable piece of fabric and the second stretchable piece of fabric is a lycra blend.

In an embodiment, the gel cap includes a stitching running throughout the length of the region having the predefined width to aid in the isolation of the first chamber and the second chamber. The stitching is substantially located in line to coronal suture of the user's skull/head. The predefined width of the region isolating the first chamber and the second chamber is at least one of: ¼ of an inch, ½ an inch, or an inch.

In an embodiment, the first mesh cover and the second mesh cover are at least a lycra blend material, a mesh fabric conventionally known in the art, material same as the first stretchable piece of fabric and the second stretchable piece of fabric.

In an embodiment, the cut-out section of the first chamber is V-shaped.

According to another embodiment, a gel cap includes a first chamber defined by a first stretchable piece of fabric, the first chamber is filled with a temperature variable substance, wherein when in use, the first chamber substantially covers a frontal region of a user's head, a front face of the user extending until cheekbones and a rear region of the user's head extending from parietal to occipital region of the user's head; and a second chamber, and a third chamber formed by a second stretchable piece of fabric, and a third stretchable piece of fabric and filled with the temperature variable substance, wherein, when in use, the second chamber and the third chamber substantially cover left-hand side and the right-hand side of the user's head except for the ears of the user. The first chamber further comprises a cut-out section conforming to the nose of the user so as not to obstruct the breathing of the user.

In an embodiment, the second chamber, and the third chamber are isolated from the first chamber by a region having a predefined width (W).

In an embodiment, a first mesh cover and a second mesh cover integrally formed in the second chamber, and the third chamber to substantially cover ears of the user on either side; and

According to another embodiment, a gel cap includes a first chamber defined by a first stretchable piece of fabric, the first chamber is filled with a temperature variable substance, wherein, when in use, the first chamber substantially covers a frontal region of a user's head, a front face of a user extending until cheekbones of the user, and top region of the head including coronal region; a second chamber formed by a second stretchable piece of fabric filled with the temperature variable substance, wherein, when in use, the second chamber substantially covers a rear region of the user's head extending from parietal to occipital region of the user's head, and a portion of left-hand side and right-hand side of the user's head.

In an embodiment, the first chamber is isolated from the second chamber by a region having a predefined width (W) extending from a first mesh cover to a second mesh cover, wherein each of the first and second mesh covers attaches the first chamber to the second chamber and substantially cover ears of the user on either side. The first chamber further comprises a cut-out section conforming to the nose of the user so as not to obstruct the breathing of the user.

These and other features and advantages along with other embodiments of the present invention will become apparent from the detailed description below, in light of the accompanying drawings.

### BRIEF DESCRIPTION OF DRAWINGS

In the drawings:

FIG. 1 is a perspective view of a gel cap in an open position shown in a wearable state, in accordance with one embodiment of the present invention.

FIG. 2 is a front view of the gel cap of FIG. 1.

FIG. 3 is a rear view of the gel cap of FIG. 1.

FIG. 4 is a perspective view of the gel cap of FIG. 1 in a closed position.

FIG. 5 is a sectional view of the gel cap of FIG. 4 taken along 5-5.

FIG. 6 shows the stretchability of the gel cap of FIG. 1.

FIG. 7 is a perspective view of a gel cap in an open position shown in a wearable state, in accordance with another embodiment of the present invention.

FIG. 8 is a front view of the gel cap of FIG. 7.

FIG. 9 is a rear view of the gel cap of FIG. 7.

FIG. 10 is a perspective view of a gel cap in an open position shown in a wearable state, in accordance with yet another embodiment of the present invention.

FIG. 11 is a front view of the gel cap of FIG. 10.

FIG. 12 is a rear view of the gel cap of FIG. 10.

### DETAILED DESCRIPTION

Some embodiments, illustrating its features, will now be discussed in detail. The words “comprising,” “having,” “containing,” “including,” “consisting,” and other forms thereof, are intended to be equivalent in meaning and be open-ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments in the description and drawings are merely exemplary.

References to “one embodiment”, “an embodiment”, “another embodiment”, “an example”, “another example”, “some embodiment”, “yet another embodiment”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment. Unless stated otherwise, terms such as “first”, “second”, and “third”, are used to arbitrarily distinguish between the elements such terms describe. Thus, these terms are not necessarily intended to indicate temporal or other prioritization of such elements. The terms pertaining to the brain/skull of the human head as used herein are used to better describe the positions of the human head that the proposed cap covers. No further details are provided on various anatomical terms used herein such as “coronal suture”, “coronal region”, “parietal”, “occipital”, and so on considering these terms are well understood by person skilled in the art.

Before describing the present invention in detail, it should be observed that the present invention relates to a gel cap that relieves headaches, migraine, headaches, fever, stress, or similar medical conditions of a user of the cap. Accordingly, the components have been represented, showing only specific details that are pertinent for an understanding of the present invention so as not to obscure the disclosure with details that will be readily apparent to those with ordinary skill in the art having the benefit of the description herein. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting but as a representative basis for teaching one skilled in the art to variously employ the present invention in virtually any appropriately detailed structure. Further, the terms and phrases used herein are not intended to be limiting but rather to provide an understandable description of the invention.

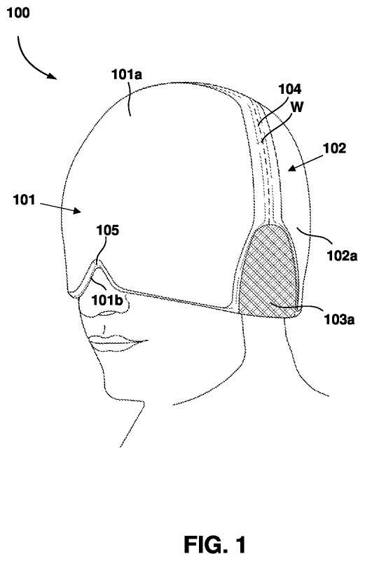

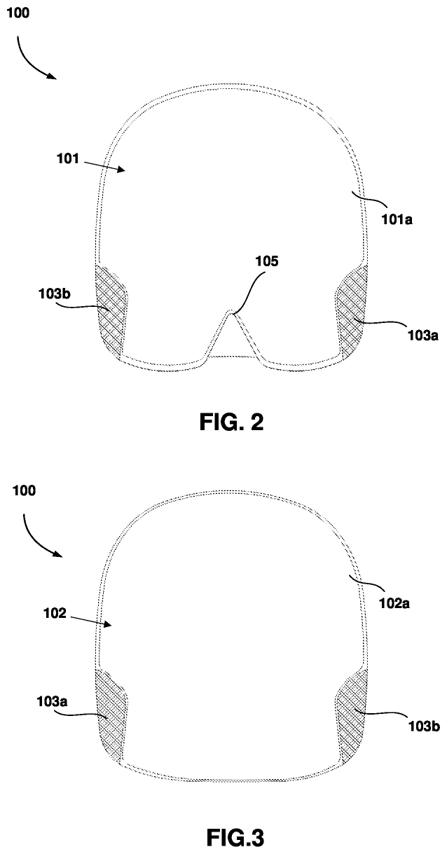

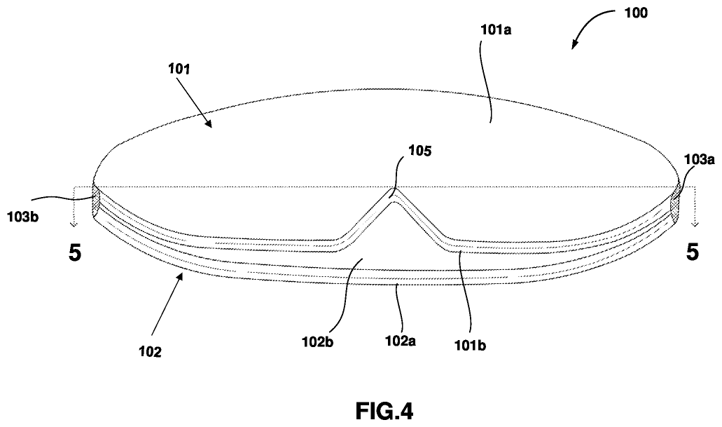

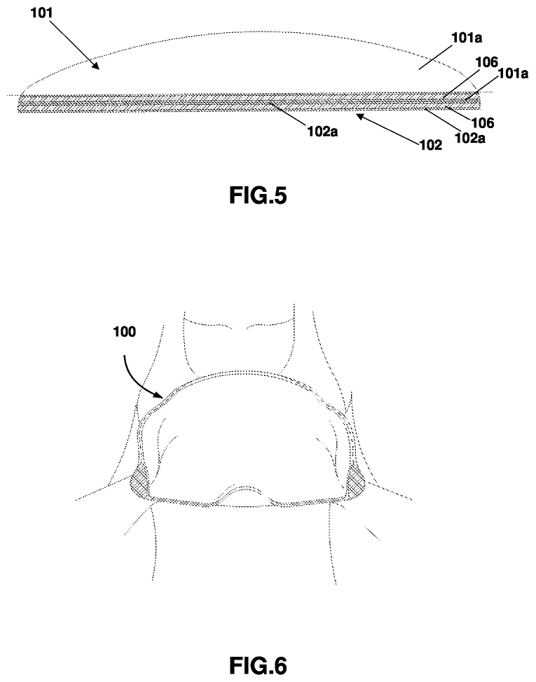

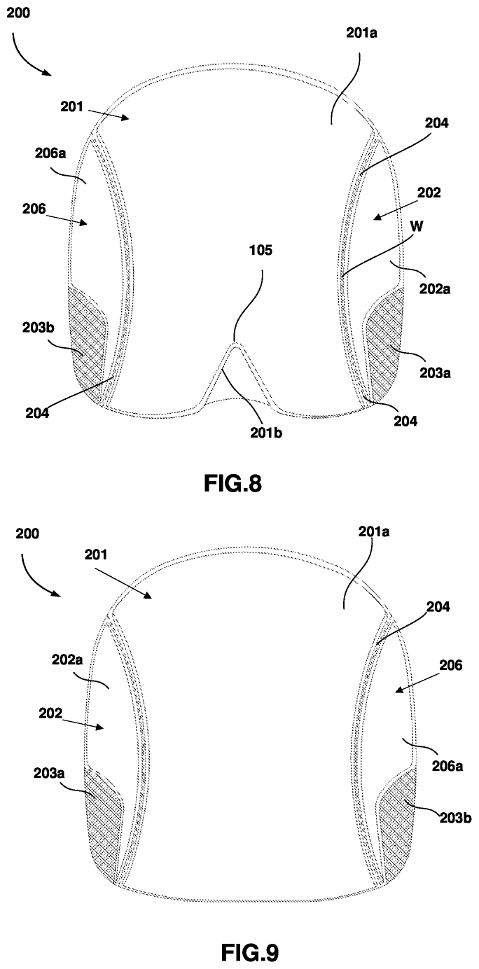

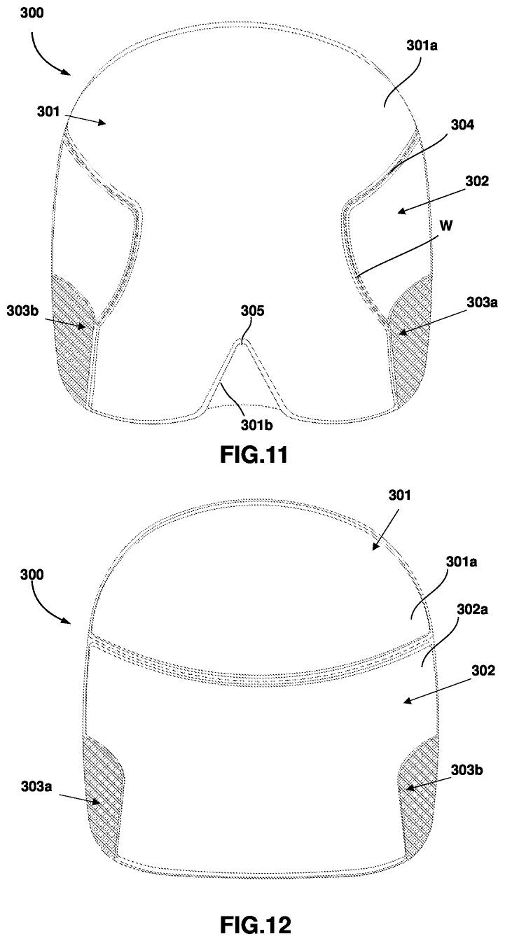

The various features and embodiments of the gel cap wearable by a user or patient are better explained in conjunction with FIGS. 1-12.

Referring to the accompanying figures, particularly FIGS. 1-6, a first embodiment of the present invention in the form of a gel cap 100 is shown.

As seen, the gel cap 100 includes one or more chambers, namely a first chamber 101 and a second chamber 102. The first chamber 101 is made or formed using a first stretchable piece of fabric 101a. The first stretchable piece of fabric 101a forms the top and bottom walls of the chamber 101. In an embodiment, the first stretchable piece of fabric 101a is a lycra blend. In some other embodiments, other suitable fabrics having the ability to stretch may be used. The chamber 101 embodies a temperature variable substance. The temperature variable substance in an embodiment is a malleable soft-solid gel. The first chamber is suitably shaped and sized such that when in use, the first chamber 101 substantially covers a frontal region of a user's head and a front face of a user extending until the cheekbones of the user.

Further, as seen, the second chamber 102 is formed or made using a second stretchable piece of fabric 102a. The second stretchable piece of fabric 102a forms the top and bottom walls of the chamber 102. In an embodiment, the second stretchable piece of fabric 102a is a lycra blend. In some other embodiments, other suitable fabrics having the ability to stretch may be used. In some embodiments, the second stretchable piece of fabric 102a may be identical to the first stretchable piece of fabric 101a. In some other embodiment, the second stretchable piece of fabric 102a may be unidentical to the first stretchable piece of fabric 101a. In some other embodiment, the second stretchable piece of fabric 102a and the first stretchable piece of fabric 101a may be made using a single piece of fabric The chamber 102 embodies a temperature variable substance. The temperature variable substance in an embodiment is a malleable soft-solid gel. The second chamber 102 is suitably shaped and sized such that when in use, the second chamber substantially covers a rear region of the user's head extending from parietal to occipital region of the user's head.

According to an embodiment, the first chamber 101 and the second chamber 102 along with the temperature variable substance are curved and streamlined so that the gel cap 100 comfortably fits around the front face of the user extending until the cheekbones and around the rear region of the head. Further, the edges of the first stretchable piece of fabric 101a forming the first chamber 101 and the second stretchable piece of fabric 102a forming the second chamber 102 are laser cut.

Further, as seen, the first chamber 101 is isolated or spaced apart from the second chamber 102 by a region having a predefined width W. The isolating region extends from a first mesh cover 103a to a second mesh cover 103b. In an embodiment, the isolating region is kept minimal in width. The width W may vary from ¼ of an inch to an inch. A stitching line 104 (sewing line) runs throughout the length of the region (preferably midway but not limited to) to aid in the isolation of the first chamber 101 and the second chamber 102. The stitching 104 is substantially located in line to the coronal suture of the user's skull/head.

According to an embodiment, as seen, the first and second mesh covers 103a, 103b attaches the first chamber 101 to the second chamber 102. The mesh covers 103a, and 103b are shaped to substantially cover the ears of the user on either side. The mesh covers 103a and 103b are at least a lycra blend material, a mesh fabric conventionally known in the art, material the same as the first stretchable piece of fabric 101a and the second stretchable piece of fabric 102a.

According to an embodiment, as seen, the first chamber 101 further includes a cut-out section 105 shaped preferably conforming to the nose of the user so as not to obstruct the breathing of the user. The cut-out section 105 of the first chamber 101 is V-shaped.

The use of stretchable fabrics 101a, 102a the gel cap 100 offers stretchability (FIG. 6) which allows the cap to fit around different head sizes.

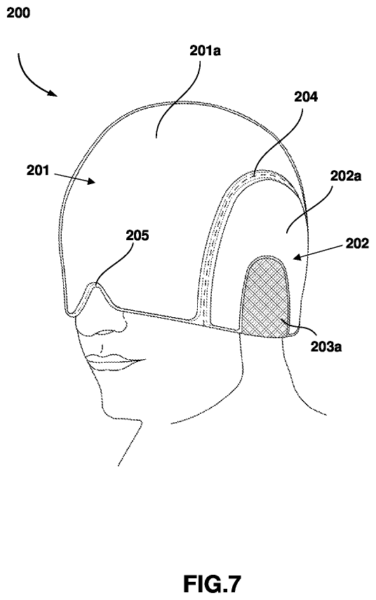

Referring to FIGS. 7-9, another embodiment of the present invention (gel cap 100) is shown. This embodiment essentially offers similar benefits as that of the embodiment shown in FIGS. 1-6, however, the design characteristics are different for the cap shown in this embodiment than the cap 100 discussed above. The cap thus shown in this embodiment will be referred to as gel cap 200.

In this embodiment, the gel cap 200 includes a first chamber 201, a second chamber 202, and a third chamber 206, in contrast to the cap design shown in FIGS. 1-6. The first chamber 201 is defined or formed by a first stretchable piece of fabric 201a. Likewise, the second and third chambers 202, and 206 are made or formed using a second stretchable piece of fabric 202a, and a third stretchable piece of fabric 206a. The stretchable piece of fabrics 201a, 202a, and 206a are of lycra blend or any other material. The stretchable pieces of fabrics 201a, 202a, 206a may all be identical or unidentical based on the design choice, however, they should exhibit stretchability. The first, second, and third chambers are filled with temperature variable substances preferably including but not limited to a malleable soft-solid gel.

In use, the first chamber 201 substantially covers a frontal region of a user's head, a front face of the user extending until cheekbones, and a rear region of the user's head extending from parietal to occipital region of the user's head, and the second chamber 202 and the third chamber 206 substantially cover left-hand side and the right-hand side of the user's head except for the ears of the user.

Like in the prior embodiment described with reference to FIGS. 1-6, the second chamber 202, and the third chamber 206 are isolated from the first chamber 201 by a region having a predefined width (W). The predefined width (W) of the region isolating the second chamber 202 and the third chamber 206 from the first chamber 201 is at least one of: ¼ of an inch, ½ an inch, or an inch. A stitching 204 running throughout the length of the region having the predefined width W is further present to aid in isolation of the second chamber 202 and the third chamber 206 from the first chamber 201. The region or stitching 140 around the region runs substantially around the temporal region of the user's head/skull.

The gel cap 200 in this embodiment also includes a first mesh cover 203a and a second mesh cover 203b integrally formed (by stitching for example) in the second chamber 202, and the third chamber 206 and suitably located to substantially cover ears of the user on either side.

The gel cap 200 in this embodiment also further comprises a cut-out section 205 conforming to the nose of the user so as not to obstruct the breathing of the user. The cut-out section 205 of the first chamber 201 is V-shaped.

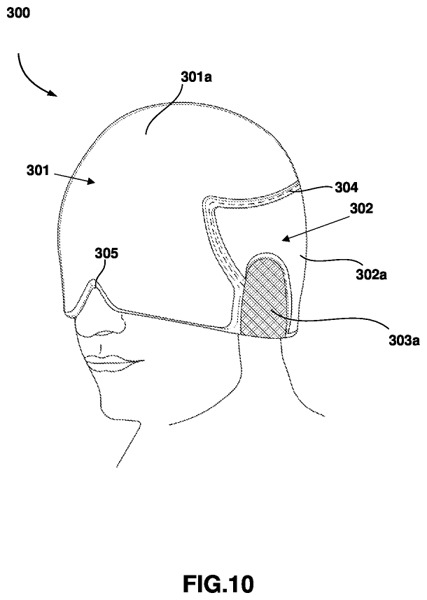

Referring to FIGS. 10-12, another embodiment of the present invention (gel cap 100) is shown. This embodiment essentially offers similar benefits as that of the embodiment shown in FIGS. 1-6, and FIGS. 7-9, however, the design characteristics are different for the cap shown in this embodiment than the caps 100, 200 discussed above. The cap thus shown in this embodiment will be referred to as gel cap 300.

In this embodiment, the gel cap 300 includes a first chamber 301, a second chamber 302, in contrast to the cap with three chambers shown in FIGS. 7-9. The first chamber 301 is defined or formed by a first stretchable piece of fabric 301a. Likewise, the second chamber 302 is made or formed using a second stretchable piece of fabric 302a. The stretchable piece of fabrics 301a, 302a are of lycra blend or any other material. The stretchable pieces of fabrics 301a, 302a may all be identical or unidentical based on the design choice, however, they should exhibit stretchability. The first, and second, chambers are filled with temperature variable substances preferably including but not limited to a malleable soft-solid gel.

In use, the first chamber 301 substantially covers a frontal region of a user's head, a front face of a user extending until cheekbones of the user, and the top region of the head including the coronal region, and the second chamber 302 substantially covers a rear region of the user's head extending from parietal to occipital region of the user's head, and a portion of left-hand side and right-hand side of the user's head.

Like in the prior embodiments described with reference to FIGS. 1-6, and 7-9, the first chamber 301 is isolated from the second chamber 302 by a region having a predefined width (W) extending from a first mesh cover 303a to a second mesh cover 303b. Each of the first and second mesh covers 303a, 303b attaches the first chamber 301 to the second chamber 302 and substantially covers ears of the user on either side. Further, a stitching line 304 runs throughout the length of the region having the predefined width (W) to aid in the isolation of the second chamber 302 from the first chamber 301. The predefined width (W) of the region isolating the second chamber (302) from the first chamber (301) is at least one of: ¼ of an inch, ½ an inch, or an inch.

The gel cap 200 in this embodiment also further comprises a cut-out section 305 conforming to the nose of the user so as not to obstruct the breathing of the user. The cut-out section 305 of the first chamber 301 is V-shaped.

The caps 100, 200, 300 are useful for the application of both hot and cold therapy. Typically, the caps 100, 200, 300 can be kept in the freezer for a certain time (say a couple of hours) and once they are cool, one can open and wear the cap to get relief from different medical conditions say headache, migraine, fever, etc. Likewise, the caps 100, 200, 300 can be kept in the microwave or other product-compatible heating source for certain time to heat up the cap to the desired temperature, and once heated, one can wear the cap to get relief from different medical conditions such as for example, during sinus problem.

Although particular embodiments of the invention have been described in detail for purposes of illustration, various modifications and enhancements may be made without departing from the spirit and scope of the invention.

What is claimed is:

1. A gel cap (100), comprising:
a first chamber (101) defined by a first stretchable piece of fabric (101a), the first chamber (101) is filled with a temperature variable substance, wherein, when in use, the first chamber (101) substantially covers a frontal region of a user's head and a front face of a user extending until cheekbones of the user;
a second chamber (102) formed by a second stretchable piece of fabric (102a) filled with the temperature variable substance, wherein, when in use, the second chamber (102) substantially covers a rear region of the user's head extending from parietal to occipital region of the user's head;
wherein the first chamber (101) is isolated from the second chamber (102) by a region having a predefined width (W) extending from a first mesh cover (103a) to a second mesh cover (103b), wherein each of the first and second mesh covers (103a, 103b) attaches the first chamber (101) to the second chamber (102) and substantially cover ears of the user on either side; and
wherein, the first chamber (101) further comprises a cut-out section (105) conforming to the nose of the user so as not to obstruct the breathing of the user.

2. The gel cap (100) of claim 1, wherein the temperature variable substance is a malleable soft-solid gel.

3. The gel cap (100) of claim 1, wherein the first stretchable piece of fabric (101a) and the second stretchable piece of fabric (102a) are identical or unidentical.

4. The gel cap (100) of claim 1, wherein the first stretchable piece of fabric (101a) and the second stretchable piece of fabric (102a) is a single one-piece fabric.

5. The gel cap (100) of claim 1, wherein the first stretchable piece of fabric (101a) and the second stretchable piece of fabric (102a) is a lycra blend.

6. The gel cap (100) of claim 1, wherein the first chamber (101) and the second chamber (102) along with the temperature variable substance are curved and streamlined so that the gel cap (100) fits better around the front face of the user extending until the cheekbones and around the rear region of the head.

7. The gel cap (100) of claim 1, wherein the edges of the first stretchable piece of fabric (101a) and the second stretchable piece of fabric (102a) are laser cut.

8. The gel cap (100) of claim 1 further comprises a stitching (104) running throughout the length of the region having the predefined width (W) to aid in the isolation of the first chamber (101) and the second chamber (102).

9. The gel cap (100) of claim 1, wherein the stitching (104) is substantially located in line to coronal suture of the user's skull.

10. The gel cap (100) of claim 1, wherein the first mesh cover (103a) and the second mesh cover (103b) are at least a lycra blend material, a mesh fabric conventionally known in the art, material same as the first stretchable piece of fabric (101a) and the second stretchable piece of fabric (102a).

11. The gel cap (100) of claim 1, wherein the predefined width (W) of the region isolating the first chamber (101) and the second chamber (102) is at least one of: ¼ of an inch, ½ an inch, or an inch.

12. The gel cap (100) of claim 1, wherein the cut-out section (105) of the first chamber (101) is V-shaped.

13. A gel cap (200), comprising:
a first chamber (201) defined by a first stretchable piece of fabric (201a), the first chamber (201) is filled with a temperature variable substance, wherein when in use, the first chamber (201) substantially covers a frontal region of a user's head, a front face of the user extending until cheekbones, and a rear region of the user's head extending from parietal to occipital region of the user's head;
a second chamber (202), and a third chamber (206) formed by a second stretchable piece of fabric (202a), and a third stretchable piece of fabric (206a) and filled with the temperature variable substance, wherein, when in use, the second chamber (202) and the third chamber (206) substantially cover left hand side and the right-hand side of the user's head except for the ears of the user;
wherein the second chamber (202), and the third chamber (206) are isolated from the first chamber (201) by a region having a predefined width (W);
a first mesh cover (203a) and a second mesh cover (203b) integrally formed in the second chamber (202), and the third chamber (206) to substantially cover ears of the user on either side; and
wherein, the first chamber (201) further comprises a cut-out section (205) conforming to the nose of the user so as not to obstruct the breathing of the user.

14. The gel cap (100) of claim 13, wherein the first stretchable piece of fabric (201a), the second stretchable piece of fabric (202a), and the third stretchable piece of fabric (206a) are identical or unidentical.

15. The gel cap (100) of claim 13, wherein the temperature variable substance is a malleable soft-solid gel.

16. The gel cap (100) of claim 13, wherein the first stretchable piece of fabric (201a), the second stretchable piece of fabric (202a), and the third stretchable piece of fabric (206a) is a lycra blend.

17. The gel cap (100) of claim 13 further comprises a stitching (204) running throughout the length of the region having the predefined width (W) to aid in isolation of the second chamber (202) and the third chamber (206) from the first chamber (201).

18. The gel cap (100) of claim 13, wherein the predefined width (W) of the region isolating the second chamber (202) and the third chamber (206) from the first chamber (201) is at least one of: ¼ of an inch, ½ an inch, or an inch.

19. The gel cap (100) of claim 13, wherein the cut-out section (205) of the first chamber (201) is V-shaped.

20. A gel cap (300), comprising:
a first chamber (301) defined by a first stretchable piece of fabric (301a), the first chamber (301) is filled with a temperature variable substance, wherein, when in use, the first chamber (301) substantially covers a frontal region of a user's head, a front face of a user extending until cheekbones of the user, and top region of the head including coronal region;
a second chamber (302) formed by a second stretchable piece of fabric (302a) filled with the temperature variable substance, wherein, when in use, the second chamber (302) substantially covers a rear region of the user's head extending from parietal to occipital region of the user's head, and a portion of left-hand side and right-hand side of the user's head;
wherein the first chamber (301) is isolated from the second chamber (302) by a region having a predefined width (W) extending from a first mesh cover (303a) to a second mesh cover (303b), wherein each of the first and second mesh covers (303a, 303b) attaches the first chamber (301) to the second chamber (302) and substantially cover ears of the user on either side; and
wherein, the first chamber (301) further comprises a cut-out section (305) conforming to the nose of the user so as not to obstruct the breathing of the user.

21. The gel cap (100) of claim 20, wherein the first stretchable piece of fabric (301a), the second stretchable piece of fabric (302a) are identical or unidentical.

22. The gel cap (100) of claim 20, wherein the temperature variable substance is a malleable soft-solid gel.

23. The gel cap (100) of claim 20, wherein the first stretchable piece of fabric (301a), and the second stretchable piece of fabric (302a) are lycra blend.

24. The gel cap (100) of claim 20 further comprises a stitching (304) running throughout the length of the region having the predefined width (W) to aid in isolation of the second chamber (302) from the first chamber (301).

25. The gel cap (100) of claim 20, wherein the predefined width (W) of the region isolating the second chamber (302) from the first chamber (301) is at least one of: ¼ of an inch, ½ an inch, or an inch.

26. The gel cap (100) of claim 20, wherein the cut-out section (305) of the first chamber (301) is V-shaped.
